(19) United States
(12) Patent Application Publication
(10) Pub. No.: US 2023/0133954 A1
(43) Pub. Date: May 4, 2023

(54) ANTIMICROBIAL FOAM ARTICLES

(71) Applicant: Medtronic, Inc., Minneapolis, MN (US)

(72) Inventors: Valentino J. Tramontano, Brockton, MA (US); Kevin P. Trainor, Greenville, RI (US)

(21) Appl. No.: 17/516,262

(22) Filed: Nov. 1, 2021

(51) Int. Cl. A61K 9/12; A61K 33/38; A61K 31/155

(52) U.S. Cl. CPC A61K 9/122; A61K 33/38; A61K 31/155

(57) ABSTRACT

In some examples, an antimicrobial article including a foam member. The foam member includes a polymer matrix and at least one antimicrobial agent encapsulated in a water permeable polymer. The polymer matrix of the foam member defines a plurality of void volumes. In some examples, the antimicrobial article including the foam member may be used in a wound dressing application.

### TECHNICAL FIELD

The present disclosure relates to, in some examples, antimicrobial foam articles and techniques for making antimicrobial foam articles.

### BACKGROUND

Medical devices and wound care materials can be a source of microbial infection in a patient. For example, material used to pack or cover a wound can introduce microbes that can cause infection.

### SUMMARY

In some examples, the disclosure is directed to articles including foam members, such as wound dressings, and method for making and using the same. The foam member may include a matrix and antimicrobial agents encapsulated by a water permeable polymer. In some examples, the foam members may allow for an antimicrobial article with a relatively high loading of antimicrobial agents and/or elution of the antimicrobial agent over a relatively long period of time.

In some aspects, the disclosure is directed to an antimicrobial article comprising a foam member including a polymer matrix and at least one antimicrobial agent encapsulated in a water permeable polymer, wherein the polymer matrix of the foam member defines a plurality of void volumes.

In another aspect, the disclosure is directed to a method of manufacturing an antimicrobial article, the method comprising forming a foam member from a polymer matrix and at least one antimicrobial agent encapsulated in a water permeable polymer matrix, wherein the polymer matrix of the foam member defines a plurality of void volumes.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is conceptual schematic diagram illustrating an example antimicrobial article from a perspective view.

FIG. 2A is conceptual schematic diagram view illustrating an example antimicrobial composition in a liquid solvent contained in a vessel from a perspective view.

FIG. 2B is a conceptual schematic diagram illustrating an example antimicrobial foam article formed according to an example technique from a perspective view.

FIG. 3 is a conceptual schematic diagram illustrating an example antimicrobial article including a foam member and an adhesive layer from a side view.

FIG. 4 is a conceptual schematic diagram illustrating an example antimicrobial article with a pore size gradient from a side view.

FIG. 5 is a conceptual schematic diagram illustrating an example antimicrobial article with an alternative pore size gradient from a side view.

FIG. 6 is a flow diagram illustrating an example technique.

FIG. 7 is a chart showing the release profile of silver from an article in accordance with the present disclosure.

FIG. 8 is a chart illustrating the results of bacterial challenge testing for articles in accordance with the present disclosure.

Like symbols in the drawings indicate like elements.

### DETAILED DESCRIPTION

As described above, examples of the disclosure are directed to articles including foam members, such as wound dressings, and method for making and using the same.

Antimicrobial agents may be used in a variety of applications to kill microorganisms or otherwise stop the growth of microorganisms, such as bacteria, fungus, and viruses. In some examples, antimicrobial agents may be applied to medical devices in the form of an antimicrobial coating or thin film to provide antimicrobial properties to the device surfaces. Additionally, antimicrobial agents may be used for infection control in the treatment of patient wounds, e.g., with antimicrobial ointments or wound dressings. Chronic wounds that exist in patients are difficult to completely heal and are often present in the patient for a time frame of months or years. However, in some instances such as chronic wound care, the antimicrobial ointment/dressings may not provide for relatively long-term efficacy without requiring the frequent replacement ointment/dressings. Additionally, patient activities such as bathing or showering may require replacement of ointment/dressings, e.g., due to the saturation of a wound dressing with water or the washing away of an antimicrobial ointment.

In some examples, an antimicrobial article according to the present disclosure may address one or more needs such as those described above through the use of an antimicrobial article including a foam member. The foam member may include a polymer matrix and at least one antimicrobial agent encapsulated in a water permeable polymer. in some examples, such an antimicrobial article may have efficacy over a long-term in providing antimicrobial properties to address a need in patients with chronic wounds by providing a wound dressing with antimicrobial efficacy over a long-term, reducing the frequency of required wound dressing changes. Reducing the frequency of required wound dressing changes may reduce disturbance of the wound site and promote wound healing. An antimicrobial article according to the present disclosure may be effective even after exposure to moisture, which may allow a person wearing the antimicrobial article as a wound dressing or wearable patch to bathe or shower without needing to change the wound dressing or wearable patch.

In some examples, the articles including an antimicrobial foam in accordance with examples of the disclosure may provide for controlled release of silver ion or other antimicrobial agent from a foam member, e.g., over a relatively long period of time and/or even after being saturated with a liquid such as water. The antimicrobial agent may provide broad-spectrum efficacy against many types of microbes, also called microorganisms. An antimicrobial article which includes a foam member having a polymer matrix and at least one antimicrobial agent may have long-term efficacy and may perform similarly or outperform conventional antimicrobial foam articles. The foam member may include at least one antimicrobial agent encapsulated in a water-permeable polymer which forms the polymer matrix.

In some examples, an antimicrobial article including foam member with a polymer matrix and a plurality of void volumes may provide a useful wound dressing. In some examples, a wound dressing according to the present disclosure may include an antimicrobial article which is used to pack a wound. In some examples, packing a wound with a wound dressing according to the present disclosure may include inserting at least a portion of the antimicrobial foam article below the outer surface of the skin, optionally in combination with other wound packing materials such as gauze, solutions, gels, ointments, or the like. In some examples, a wound dressing according to the present disclosure may include an adhesive and the wound dressing may be laminated to a patient's skin. In some examples, a wound dressing according to the present disclosure may be brought into contact or nearly brought into contact with a wound site by a medical wrap. The antimicrobial article may absorb exudate from a wound. In this way, an antimicrobial article according to the present disclosure may assist in managing moisture and/or exudate near a wound site to reduce or eliminate maceration.

Antimicrobial foam articles according to the present disclosure may have antimicrobial efficacy. Antimicrobial efficacy may be defined as antimicrobial properties which kill or stop the growth or slow the growth of at least one microbe. The at least one microbe may include one or more of common microbes of clinical concern. The at least one microbe may include Staphylococcus Aureus, Pseudomonas, or Candida Yeast, or the like. In some examples, antimicrobial articles according to the present disclosure may have antimicrobial efficacy over a long-term. Antimicrobial efficacy over a long-term may be shown by bacterial challenge testing. The antimicrobial foam article may be aged for a certain period, such as one week, or such as 3 months, or such as 18 months in some examples, and then inoculated with one or more microbes. A total kill or a reduction of microbe colonies may illustrate antimicrobial efficacy, such as a 5 log reduction of colonies.

Antimicrobial foam articles according to the present disclosure may be created according to example techniques. In some examples, an antimicrobial formulation is generated by milling a water permeable polymer dissolved in a liquid medium with at least one antimicrobial agent. The antimicrobial agent may be insoluble in the liquid medium such that the at least one water permeable polymer at least partially encapsulates the at least one antimicrobial agent after the milling process. In some example techniques, an antimicrobial foam member is formed from the antimicrobial formulation. In some examples, an antimicrobial foam member is formed by applying vacuum or temperature to the antimicrobial formulation to evaporate the liquid medium. Optionally, in some examples, an adhesive agent may be applied to the antimicrobial foam member to form an antimicrobial article. Optionally, in some examples, a film may be added to the antimicrobial article. In some examples, the antimicrobial article may be applied to a target surface to provide an antimicrobial effect to the target surface. In some examples, the antimicrobial article is packed into a target area to provide an antimicrobial effect to the target area.

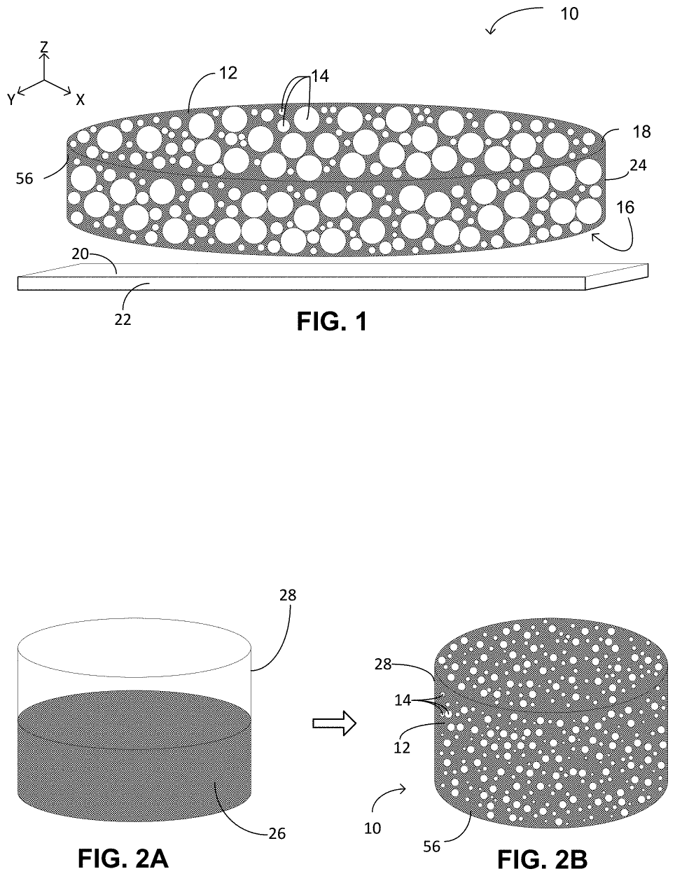

Referring to FIG. 1, example antimicrobial article 10 is illustrated in schematic perspective view relative to a substrate 22. As shown, article 10 includes foam member 56 that includes antimicrobial agent(s) (not shown individually in FIG. 1) within polymer matrix 12.

Foam member 56 of antimicrobial article 10 may have any desirable geometric shape. In some examples, foam member 56 of antimicrobial article 10 is shaped to dress or pack a wound or wound site in a patient. In some examples, such as when foam member 56 of antimicrobial article 10 is laminated to the skin as a wound dressing or wearable patch, foam member 56 may have greater length in the X and Y directions than in the Z direction. Foam member 56 may have a first surface 16 and a second surface 18. In some examples, first surface 16 is applied to a target surface 20 of a substrate 22 to provide an antimicrobial effect to target surface 20. In some examples, target surface 20. In some examples, such as when the antimicrobial article is a wound dressing packing a wound, a portion of each of first surface 16 and second surface 18 may be in contact or nearly in contact with target surface 20 and a second target surface (not shown) to provide an antimicrobial effect to one or both target surfaces, or to a target area near the target surfaces.

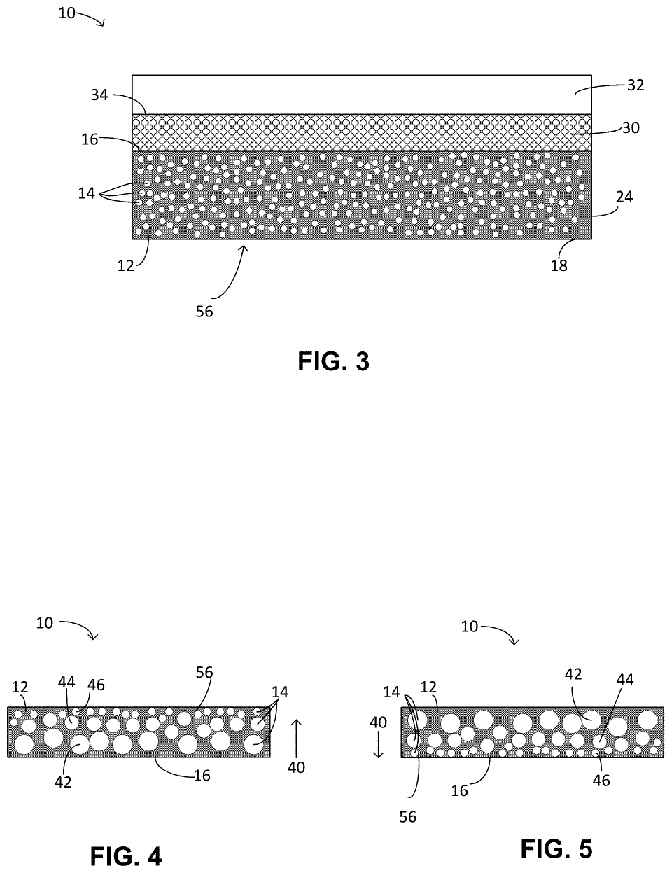

In the examples of FIG. 1, antimicrobial article 10 consists or consist essentially of foam member 56, which may include antimicrobial agent(s) in polymer matrix 12, where the polymer matrix defines a plurality of void volumes 14. In some examples, foam member 56 may be antimicrobial article 10. In other examples, antimicrobial article 10 may include foam member 56 and additional components or layers, such as adhesive layers or films, such as the example of FIG. 3.

In polymer matrix 12, foam member 56 may include at least one antimicrobial agent encapsulated (e.g., substantially fully or partially encapsulated) in a water permeable polymer. In examples, polymer matrix 12 encapsulates the at least one antimicrobial agent such that the water permeable polymer covers substantially all exterior surfaces of a particle or agglomeration of particles of the antimicrobial agent. In examples, polymer matrix 12 only partially encapsulates the at least one antimicrobial agent such that the water permeable polymer covers a portion of the exterior surfaces of a particle or an agglomeration of particles of the antimicrobial agent. In examples, polymer matrix 12 encapsulates a portion of the at least one antimicrobial agent, and a portion of the at least one antimicrobial agent is disposed within plurality of void volumes 14. In some examples, the silver sulfadiazine or other antimicrobial agent, once encapsulated by a water permeable polymer, will have portions of particles exposed at the outer surface of foam member 56. Encapsulating the antimicrobial agent in polymer matrix 12 may be desirable, as encapsulation may help to control the release of the antimicrobial agent over a long term to maintain antimicrobial efficacy of antimicrobial foam article 10.

Foam member 56 may be formed from an antimicrobial formulation which includes at least one water permeable polymer including dispersed particles (e.g., substantially uniformly dispersed) of at least one antimicrobial agent. Suitable antimicrobial formulations may include one or more of those antimicrobial formulations described in PCT Patent Application No. PCT/US2018/062218, filed Nov. 21, 2018 and U.S. patent application Ser. No. 14/567,183, filed Dec. 11, 2014, the entire content of each of these applications is incorporated herein by reference.

In some examples, foam member 56 may include a polymer matrix 12 which includes a water permeable polymer, which in turn encapsulates or partially encapsulates at least one antimicrobial agent. In other words, in some examples foam member 56 may include polymer matrix 12 as the structural foam material and the water permeable polymer as the polymer which encapsulates (e.g., substantially fully or partially encapsulates) the at least one antimicrobial agent of foam member 56. In some examples, polymer matrix 12 and the water permeable polymer which encapsulates (e.g., substantially fully or partially encapsulates) the at least one antimicrobial agent may be separate materials. In some examples, the water permeable polymer which encapsulates or partially encapsulates the at least one antimicrobial agent may be the same material or combination of materials. Put another way, the polymer matrix 12 may be formed of the same or different material compared to that of the water permeable polymer that encapsulates the antimicrobial agent(s). For example, polymer matrix 12 may be a polyurethane and the water permeable polymer that encapsulate the antimicrobial agent(s) of foam member 56 may also be a polyurethane or a different water permeable polymer other than a polyurethane.

In some examples, polymers and/or antimicrobial agents for an antimicrobial formulation that may be useful for forming foam member 56 may be not readily soluble. In some such examples, such ingredients may tend to form agglomerated particles in formulations used to form foam member 56, ultimately leading to the presence of agglomerated particles in antimicrobial article 10, or on target surface 20. Agglomerations may refer to a bound or joined plurality of particles, individual particles of the plurality retaining their identity. Even when the initial size of the particles used in preparing foam member 56 is as small as several microns, the particles can agglomerate resulting in agglomerated particle sizes as large as several hundred microns. Such agglomerated particles may result from the agglomeration of many smaller sized particles that agglomerate during formation of foam member 56 from the antimicrobial formulation.

The size and shape of the agglomerated particles can adversely affect the dissolution or release of the antimicrobial agent from foam member 56. To avoid or minimize adverse effects, the average size of particles and agglomerates may be maintained lower than a predetermined threshold. In some examples, the particles and any agglomerations of the at least one antimicrobial agent have an average size of no greater than 50 microns. For example, foam member 56 may include particles having a mean particle size and mean agglomeration size of no greater than about 50 microns, no greater than about 40 microns, no greater than about 30 microns, no greater than about 20 microns, no greater than about 10 microns, no greater than about 5 microns, in a range from about 0.5 about 5 microns, from about 0.5 to about 20 microns, from about 0.5 to about 50 microns, from about 1 to about 10 microns, from about 1 to about 20 microns, from about 1 to about 30 microns, from about 1 to about 40 microns, from about 1 to about 50 microns, and numbers therebetween.

In some examples, the water permeable polymer in foam member 56 defines a bulk of polymer matrix 12 in which particles of antimicrobial agents are uniformly dispersed. Polymers useful for forming polymer matrix 12 include polymers that are water permeable and are formable into a foam such as, for example, a polyurethane, such as a thermoplastic polyurethane elastomer, a polyester, polylactic acid, polyglycolic acid, polytetramethylene glycol, polyacrylamide, polyacrylic acid, polyacrylate, poly(2-hydroxyethylmethacrylate), polyethylene-imine, poly-sulfonate and copolymers thereof such as poly(lactic acid-co-glycolic acid) (PLA/PGA), polyacrylic-co-hydroxylated-acrylate, poly(acrylic acid-co-2-hydroxy ethyl methacrylate). In some examples, the polymer is a thermoplastic polyurethane elastomer, such as Pellethane (Lubrizol Advanced Materials, Wickliffe, Ohio, USA).

In some examples, the weight average molecular weight of the polymer is sufficiently high enough to form a free-standing foam but not so high as to prevent a formulation of the polymer from being formed into a foam. Such polymers may have a weight average molecular weight (Mw), for example, of from about 20,000 to about 500,000 Daltons, e.g., in a range from about 50,000 to about 200,000, or from about 70,000 to about 120,000 Daltons. The weight average molecular weights can be determined by using GPC analysis having a refractive index detector coupled with a light scattering detector for absolute molecular weight measurement of weight average molecular weight (Mw).

In some examples, one or more polymers of polymer matrix 12 may provide foam member 56 with one or more of a predetermined flexibility, conformability, or elasticity to allow foam member 56 to conform and contact uniformly along target surface 20. In some examples, the predetermined flexibility, conformability, or elasticity allows antimicrobial article 10 to pack a wound.

Molecules of the antimicrobial agents may be released from the particles and may diffuse or otherwise migrate from an interior of foam member 56 to one of the major surfaces (16, 18, 24) of foam member 56. Ultimately, molecules of the antimicrobial agents may leach from antimicrobial article 10 into an adjacent or surrounding environment, providing the antimicrobial effect at or adjacent antimicrobial foam article 10.

Antimicrobial agents that may be employed in foam members, such as, foam member 56, include, for example, silver-based antimicrobial agents; polybiguanides and salts thereof; chlorhexidine and salts thereof such as the dihydrochloride, diacetate and digluconate salt of chlorhexidine; hexachlorophene; cyclohexidine; chloroaromatic compounds such as triclosan; para-chloro-meta-xylenol.

Silver-based antimicrobial agents include, for example, silver particles; silver nitrate; silver halides, e.g., silver fluoride, chloride, bromate, iodate; silver acid salts, e.g., silver acetate, silver salicylate, silver citrate, silver stearate, silver benzoate, silver oxalate; silver permanganate; silver sulfate; a silver nitrite; silver dichromate; silver chromate; silver carbonate; silver phosphate; silver (I) oxide; silver sulfide; silver azide; silver sulfite; silver thiocyanate; and silver sulfonamide, such as a silver sulfadiazine. Antimicrobial agents that are not readily soluble in an antimicrobial formulation are particularly advantageous in the present disclosure.

In some examples, the antimicrobial agents of foam member 56 include a combination of a silver-based salt and a polybiguanide salt, i.e., a combination of silver sulfadiazine and chlorhexidine diacetate. For example, the antimicrobial agent in foam member 56 may include a silver-based salt and a polybiguanide salt. In some examples, the silver-based salt is silver sulfadiazine, and the polybiguanide salt is chlorhexidine diacetate. In some examples, foam member 56 includes silver sulfadiazine in at least about 2 weight percent (wt. %), such as about 2 weight percent (wt. %) to about 10 wt. %; at least 9 wt. % of chlorhexidine diacetate, and the water permeable polymer in a range from about 70 wt. % to about 90 wt. %. In some examples, foam member 56 consists or consists essentially of the antimicrobial agents and water permeable polymer. In some examples, foam member 56 has a release profile such that at least 0.50 micrograms per centimeter (μg/cm) of silver is continuously released after 150 hours. In some examples, foam member 56 has a release profile such that at least 10 μg/cm of chlorhexidine diacetate is continuously released after about 150 hours.

Relatively high loading of at least one antimicrobial agent compared to other antimicrobial foams with lower loading, as disclosed in this and other examples, helps to allow foam member 56 to retain its antimicrobial efficacy even after exposure to moisture, such as when a patient bathes or showers. A relatively high load of at least one antimicrobial agent also helps to allow the foam member 56 to retain its antimicrobial efficacy for long periods of time. In some examples, foam member 56 includes at least 0.5% of at least one antimicrobial agent by weight of foam member 56. In some examples, foam member 56 may include at least 1 wt. % of the at least one antimicrobial agent. In some examples, foam member 56 may include at least 5 wt. % of the at least one antimicrobial agent. In some examples, foam member 56 may include at least 10 wt. % of the at least one antimicrobial agent.

In some examples, the at least one antimicrobial agent may include two distinct types of antimicrobial agents. In some examples, foam member 56 may include at least 0.5 wt. % of each of the two antimicrobial agents. In some examples, foam member 56 may include at least 1 wt. % of each of the two types of antimicrobial agents. In some examples, foam member 56 may include at least 5 wt. % of each of the two antimicrobial agents. In some examples, the at least one antimicrobial agent may include two antimicrobial agents, silver sulfadiazine and chlorhexidine acetate, and foam member 56 may comprise about 7 wt. % silver sulfadiazine and about 11.55 wt. % chlorhexidine diacetate, e.g., with the remainder being the water permeable polymer/polymer matrix 12.

The average particle size (surface area) and solubility of the antimicrobial agents and the water permeability of the polymer may affect the release rate of the antimicrobial agent from antimicrobial foam article 10. In some examples, the antimicrobial agent in foam member 56 includes silver sulfadiazine, and foam member 56 has a release profile wherein at least 0.50 μg/cm of silver is continuously released after 72 hours. In some examples, foam member 56 includes chlorhexidine diacetate, and at least 10 μg/cm of chlorhexidine diacetate is continuously released after 72 hours.

For example, relatively more water-soluble antimicrobial agents such as chlorhexidine gluconate will have a higher release rate whereas the relatively water insoluble hydrochloride salt releases slowly. In some examples, antimicrobial foam article 10 includes from about 70 wt. % to 90 wt. % of a polyurethane polymer, from 2 wt. % to about 10 wt. % silver sulfadiazine, e.g., from about 3.5 wt. % to about 7 wt. % and a minimum amount of chlorhexidine diacetate of about 9 wt. %, 10 wt. %, or about 11 wt. %. In such cases, foam member 56 can be formed to have a release profile wherein at least 0.50 μg/cm of silver is continuously released after 75 hours, e.g., after about 100 hours, 150 hours and higher, and wherein at least 10 μg/cm of chlorhexidine diacetate is continuously released after 75 hours, e.g., after about 100 hours, 150 hours and higher.

Foam member 56 includes plurality of void volumes 14. Plurality of void volumes 14 may be in the form of cells or pores. Plurality of void volumes 14 are spaces or pockets within foam member 56 that are not filled by the polymer matrix 12, e.g., which are void of any material. In some examples, void volumes 14 in foam matrix 12 are caused by solvent evaporation during a process to form foam member 56, e.g., as described herein, leaving the voids in a random pattern. These voids, or cells, may be open throughout foam member 56 and at the surface of foam member 56.

In some examples, plurality of void volumes 14 are connected to each other. In other words, respective individual void volumes of the plurality of void volumes are not completely surrounded by the polymer matrix 12, e.g., so antimicrobial foam article 10 is an open cell foam. In some examples, foam member 56 constructed from an open cell foam increases the rate of absorption of moisture or exudate, which may be desirable in instances where antimicrobial article 10 packs a wound which produces relatively high amounts of exudate or is placed on a surface or packed in an area which has relatively high amounts of moisture.

Additionally, or alternatively, in some examples, respective individual void volumes of the plurality of void volumes 14 are not connected to each other. For example, in such an instance, each void volume may be completely surrounded by polymer matrix 12, e.g., so foam member 56 is a closed cell foam. Antimicrobial article 10 comprising a closed cell foam may have reduced absorption of moisture or exudate relative to examples constructed from open cell foams, which may be desirable in instances where it is desired to leave antimicrobial article 10 in place laminated to a wound site or packing a wound for a long term.

Plurality of void volumes 14 of foam member 56 may be varied in size and quantity, e.g., to change the release characteristics of the at least one antimicrobial agent. Plurality of void volumes 14 may be substantially constant in size and shape or may vary. The relative amount of the plurality of void volumes 14 to polymer matrix 12 for foam member 56 may be expressed as a void volume percent, which may refer to the total volume of the plurality of void volumes divided by the total bulk volume (e.g., as determine by the external dimension of foam member 56) of foam member 56. Foam members 56 with a relatively high void volume percentage may increase the diffusion of the at least one antimicrobial agent from antimicrobial foam article into the surrounding environment, and thus increase the antimicrobial effect, relative to antimicrobial foam articles with lower void volume percentage, which may diffuse a small amount of the at least one antimicrobial agent into the surrounding environment over a longer time period. In some examples, foam member 56 includes about 0.5 percent void volume to about 99% void volume, such as at least about 5% void volume, such as about 5% to about 95% void volume, at least about 10%, at least about 15%, at least about 20%, at least about 30%, at least about 40%, or at least about 50% void volume. In some examples, foam member 56 may include at least about 1% void volume, such as, at least about 10% void volume.

Foam member 56 may be described in terms of total surface area of the polymer matrix 12 per volume of foam member 56, expressed, for example, in square meters per cubic meter (m²/m³). In examples where foam member 56 has a relatively high surface area, the diffusion of the at least one antimicrobial agent from antimicrobial foam article into the surrounding environment may be relatively fast, thus increase the antimicrobial effect, relative to antimicrobial foam articles with lower surface area, which may diffuse a small amount of the at least one antimicrobial agent into the surrounding environment over a longer time period.

Any suitable technique may be used to form foam member 56 in accordance with examples of the disclosure. FIGS. 2A and 2B are illustrations representative of one example technique that may be employed. The example technique of FIGS. 2A and 2B, an antimicrobial formulation in a liquid medium 26 is partially filled in vessel 28, e.g., as shown in FIG. 2A. Vessel 28 is configured to receive a volume of liquid and retain the liquid within the volume. In some examples, the antimicrobial formulation in liquid medium 26 may be substantially as described above in relation to FIG. 1. In some examples, the antimicrobial formulation may ultimately make up polymer matrix 12 after at least a portion of the liquid medium is removed (e.g., after a solvent is removed).

In some examples, the liquid medium of the present disclosure includes one or more liquids (e.g., solvents) that dissolve or suspend the polymer and/or antimicrobial agent of foam member 56, such as those described herein. Such liquids/solvents include, for example, one or more of the following: an alcohol and lower alcohol, e.g., a C1-12 alcohol, such as methanol, ethanol, n-propanol, isopropanol, n-butanol, isobutanol, furfuryl alcohol; a polyhydridic alcohol, such as ethylene glycol, a butanediol, a propanediol; an ether, such as a linear, branched or cyclic lower ether, dimethyl ether, ethyl ether, methyl ethyl ether, tetrahydrofuran (THF); a ketone such as a linear, branched or cyclic lower ketone, such as acetone, methyl ethyl ketone, cyclohexanone; an organic acid, such as formic acid, acetic acid, butyric acid, benzoic acid; an organic ester, such as a formate, ethyl or methyl acetate, propionate; an amide such as a linear, branched or cyclic lower amide, such as dimethylacetamide (DMAC), pyrrolidone, 1-Methyl-2-pyrrolidinone (NMP), a hydrocarbon, such as a linear, branched or cyclic alkane, such as a pentane, hexane, heptane, octane, cyclohexane, a linear, branched or cyclic alkene, an aromatic solvent or liquid; and a halogenated solvent or liquid such as a chlorinated solvent or liquid.

In some examples, the liquid medium 26 includes a primary alcohol, e.g., a primary C1-6 alcohol such as methanol, ethanol, n-propanol, n-butanol, n-pentanol, n-hexanol in the formulation. N-propanol may have a beneficial balance between the length of the aliphatic chain and the hydroxyl group.

Vessel 28 may have an internal cavity of any shape or size suitable to create an antimicrobial foam article. In some examples, vessel 28 may act as a vacuum chamber. In other words, vessel 28 may be constructed of materials capable of holding a seal to withstand external pressure and be outfitted with a vacuum system configured to reduce the pressure in the internal volume of vessel 28 by evacuating the atmosphere not occupied by antimicrobial formulation dissolved in liquid medium 26.

In FIG. 2A, vessel 28 is partially filled liquid medium 26 including an antimicrobial solution at least partially dissolved in a solvent. In some examples, a vacuum system may be activated which evacuates air or other atmospheric gases. FIG. 2B illustrates the system after a vacuum or partial vacuum is applied to the internal volume of vessel 28. The solvent from liquid medium 26 evaporates under vacuum, causing bubbles to form in the remaining antimicrobial formulation. Bubbles are entrained in the antimicrobial formulation as the solvent portion of liquid medium 26 is driven off, forming polymer matrix 12 with a plurality of void volumes 14. All or substantially all of the solvent from liquid medium 26 may be removed, and foam member 56 may result. Foam member 56 may be removed from vessel 28. Vessel 28 may be a mold which forms foam member 56 as a substantially finished part, which may be antimicrobial article 10. Alternatively, foam member 56 may be further processed, machined, cut, shaped, or the like.

In some examples, liquid medium 26 includes a polymer formulation with the antimicrobial agent(s) dissolved in one or more solvents, where the polymer used to form polymer matrix 12 is in its final state (fully polymerized) such as a fully polymerized polyurethane. Any heating or vacuum steps used to form foam member 56 from liquid medium 26 does not change the molecular weight of the polymer.

As noted above, in some examples, one or more other components may be added to foam member 56, e.g., in the case of a wound dressing. FIG. 3 is a schematic and conceptual side view of another example antimicrobial foam article 10 including an adhesive layer 30 on foam member 56.

Foam member 56 may be substantially the same as that that described with regard to FIG. 1. Adhesive layer 30 may be formed of an adhesive composition. In some examples, the term adhesive composition refers to a composition including at least one agent that promotes adhesion between two predetermined surfaces. In the example shown in FIG. 3, single adhesive layer 30 is formed adjacent first surface 16 of foam member 56. However, the adhesive layer 30 may be applied to one or more of major surfaces 16, 18 or 24. In some examples, adhesive layer 30 may include, instead of, or addition to, the adhesive agent, a tackifier. In some such examples, adhesive layer 30 may define the tacky surface. The adhesive agent may include any suitable adhesive formulation, for example, a pressure-sensitive adhesive, a silicone-based adhesive, or a biocompatible adhesive. The tackifier may include any suitable tacky formulation, for example, including natural or synthetic resins. In some examples, adhesive layer 30 may extend beyond the edges, such as the edge defining major surface 24 of foam member 56 so that antimicrobial article 10 may be laminated to the skin with major surface 18 of foam member 56 contacting the skin.

As shown in FIG. 3, in some examples, antimicrobial foam article 10 may include a film 32. In some examples, film 32 may substantially cover surface 34 of adhesive layer 30. In some examples, film 32 may be a release liner, which may protect the adhesive layer 30 until the antimicrobial foam article 10 is applied as a wound dressing, when film 32 is removed and discarded. In some examples, film 32 be designed to remain a part of antimicrobial article 10 during use. Film 32 may be coated with any suitable adhesive to secure the wound dressing to the skin. Film 32 may be breathable to allow moisture to evaporate from the skin. In some examples, film 32 extends with adhesive layer 30 beyond the edges of foam member 56, such as the edge defining major surface 24, so that antimicrobial article 10 may be laminated or otherwise adhered to the skin with major surface 18 of foam member 56 contacting the skin and film 32 both supports adhesive layer 30 and forms an exterior barrier. In some examples, antimicrobial foam article 10 may include foam member 56 laminated or otherwise attached to a suitable medical device tape, and antimicrobial foam article 10 may function as an adherable wound dressing, covering, patch, or the like. In some examples, foam member 56 may be applied to the area of a patient with a film dressing such as Tegaderm® (commercially available from 3M of Minnesota USA) to provide antimicrobial properties to the applied area with foam member 56.

As described above, in some examples, plurality of void 14 may be substantially constant or may vary throughout the bulk volume of foam member 56. Referring now to FIGS. 4 and 5, as illustrated in FIG. 4, foam member 56 having a plurality of void volumes 14 may have a void volume gradient 40, which may be achieved by having larger void volumes 42 toward the wound-facing side, first surface 16 of antimicrobial article 10. Antimicrobial article 10 may be used, for example, as a wound dressing. The pore size decreases (44, 46) further away from first surface 16 within foam member 56. The larger void volumes 42 at the wound facing side, first surface 16, may allow a high level of exudate into the foam which in turn can causes a correspondingly relatively high level of the at least one antimicrobial agent to be released from polymer matrix 12 into the wound initially. As more fluid is introduced and travels further into the dressing, the fluid absorption profile is affected by void volume gradient 40, with a corresponding effect on the release profile of the active agent. Thus, for example, with this configuration a relatively large amount of antimicrobial agent may be released initially with a corresponding relatively large absorption of wound exudate. Subsequently, relatively smaller amounts of the at least one antimicrobial agent released, and the correspondingly relatively smaller absorption of exudate.

In some examples, the absorption and release profile of the at least one antimicrobial agent may be reversed relative to the embodiment illustrated in FIG. 4. For example, FIG. 5 illustrates antimicrobial article 10 comprising foam member 56 with a void volume gradient 40 which is opposite the example in FIG. 4, with smaller void volumes 46 located toward first surface 16 and larger void volumes 42 further away from first surface 16, with medium-sized void volumes 44 located toward the middle of foam member 56. When used as a wound dressing, smaller void volumes 46 at first surface 16, configured to face the wound, allow a relatively low amount of wound fluid or exudate into foam member 56 initially, which in turn may cause a corresponding relatively low level of the at least one antimicrobial agent contained within polymer matrix 12 to be released into the wound initially. As more fluid is introduced and travels further into the dressing, the fluid absorption profile is affected by the void volume gradient 40, with a corresponding effect on the release profile of the at least one antimicrobial agent. Additional changes in the average size of the void volumes as fluid travels further up into foam member 56 may induce a further change in the fluid absorption and/or release profiles of the at least one antimicrobial agent. Thus, for example, with this configuration a relatively small amount of at least one antimicrobial agent may be released initially along with a corresponding relatively small absorption of wound exudate, with relatively larger amounts of at least one antimicrobial agent released subsequently, and the correspondingly relatively larger absorption of exudate.

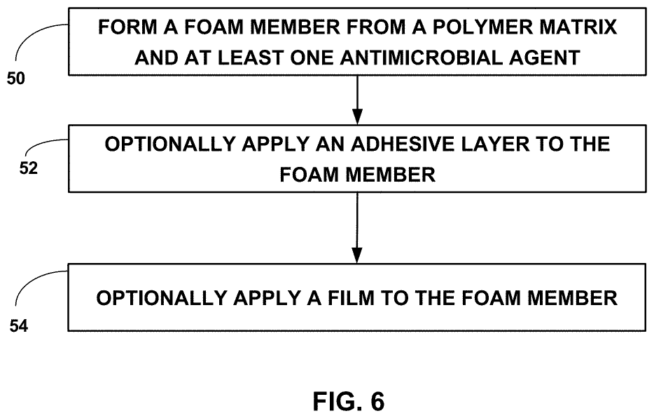

FIG. 6 is a flow diagram illustrating an example technique for forming an antimicrobial article such as article 10 including foam member 56. While the example technique of FIG. 6 is described with reference to antimicrobial article 10 to FIGS. 1-5, example techniques according to the disclosure may be used to form any antimicrobial article according to the disclosure.

As shown in FIG. 6, foam member 56 may be formed from a formulation including a polymer and at least one antimicrobial agent (50). Suitable formulation includes those described above. In some examples, the example technique of FIG. 6 includes milling the at least one water permeable polymer which is dissolved in a liquid medium with at least one antimicrobial agent which is insoluble in the liquid medium to form an antimicrobial formulation in which the at least one water permeable polymer encapsulates the at least one antimicrobial agent. In some examples, milling may completely encapsulate the at least one antimicrobial agent in the water permeable polymer which forms the polymer matrix. In some examples, milling may partially encapsulate the at least one antimicrobial agent in the water permeable polymer which forms the polymer matrix. The at least one antimicrobial agent and water permeable polymer may include any antimicrobial agent and water permeable polymer as described with reference to FIG. 1. The liquid medium may include any liquid medium as described with reference to FIGS. 2A and 2B.

In some examples, antimicrobial agents (e.g., silver sulfadiazine particles) are encapsulated (e.g., at least partially) in the water permeable polymer material (e.g., polyurethan polymer) during the milling process. The water permeable polymer may be the polymer matrix that creates matrix 12 of foam member 56 following the milling, e.g., using the process described with reference to FIGS. 2A and 2B.

Milling the at least one water permeable polymer and at least one antimicrobial agents in liquid media may offer the advantage of forming uniform antimicrobial formulations that can be used to form antimicrobial foam members. It is believed that milling, rather than mixing such as with a high shear mixer, a liquid medium including the at least one water permeable polymer with the at least one antimicrobial agent enables the at least antimicrobial agent to be uniformly dispersed in the liquid medium and/or to be encapsulated within the polymer such that the antimicrobial agent does not re-agglomerate prior to and during forming of an antimicrobial foam article. Encapsulation of the at least one antimicrobial agent in at least one water permeable polymer is believed to provide a more consistent elution rate of the at least one antimicrobial agent and prevent the re-agglomeration of antimicrobial particles over time. Milling the formulation can be carried out using a high-shear miller such as a roll mill. Milling media useful for the present disclosure include Yttria stabilized zirconia grinding media, ⅜ inch cylinder shape, from Inframat Advanced Materials.

In one aspect of the present disclosure, at least one antimicrobial agent is insoluble in the formulation and the formulation is milled until the insoluble antimicrobial agent has a mean particle size of no greater than about 50 microns, such as no greater than 40 microns, 30 microns, 20 microns, 10 microns, 5 microns and numbers therebetween. In one embodiment, the mean particle size is approximately 5 microns. Mean particle size determinations can be made by a laser diffraction particle size analyzer, such as the Microtrac S3500 with a circulating loop to suspend the sample during analysis.

Example techniques for forming an antimicrobial formulation that are subsequently used to form foam member 56 may include those techniques described in PCT Patent Application No. PCT/US2018/062218, filed Nov. 21, 2018 and/or U.S. patent application Ser. No. 14/567,183, filed Dec. 11, 2014.

In some examples, forming antimicrobial foam member 56 from a polymer matrix and at least one antimicrobial agent (50) may include evaporating the liquid medium from an antimicrobial formulation. In some examples, an antimicrobial foam article is formed by containing the antimicrobial formulation (e.g., in liquid medium 26) in a vessel and applying a vacuum or partial vacuum pressure to the vessel containing the antimicrobial formulation, thereby driving off at least a portion of the liquid medium solvent. In some examples, the vessel may be vessel 28 may be as described with reference to FIGS. 2A and 2B above. A vacuum oven may serve as a vessel in accordance with the present disclosure. In some examples, the liquid medium may be liquid medium 26 as described with reference to FIGS. 2A and 2B above. At pressure below standard atmospheric pressure, the liquid medium may rapidly evaporate, forming gas bubbles which are entrained in the antimicrobial formulation. After a substantial portion of the liquid medium is driven off, the antimicrobial formulation may form a polymer matrix with a plurality of void volumes. The polymer matrix may be the same as or similar to polymer matrix 12 according to any of the examples as described with reference to FIGS. 1-5 above. The plurality of void volumes may be the same or similar to the plurality of void volumes 14 according to any of the examples as described above with reference to FIGS. 1-5. Accordingly, the resulting antimicrobial article may the same as or similar to the antimicrobial article 10 as described above with reference to any of FIGS. 1-5.

The speed with which the vacuum or partial vacuum pressure is applied to vessel 28 may control the quantity and magnitude of bubbles formed in the antimicrobial formulation, which ultimately controls the quantity, size, and geometry of the plurality of void volumes. As vacuum is increased with substantially constant heat, a relatively larger magnitude of void volumes may be created. Similarly, as heat or energy input increases with constant vacuum, a relatively larger magnitude of void volumes may be created.

In some examples, forming an antimicrobial foam article from the antimicrobial formulation may involve heating the antimicrobial formulation to rapidly evaporate the liquid medium. In some examples, forming an antimicrobial foam article from the antimicrobial foam article may include mixing air or another gas or mixture of gases with the antimicrobial formulation to form a foam via a dispersion process. In some examples, forming an antimicrobial foam may include a reactive process such as, e.g., for example, the use of a chemical that generated carbon dioxide in the antimicrobial formulation/liquid medium 26 where the bubbling action in the liquid would form a foam.

In some examples, the process of FIG. 6 optionally includes applying an adhesive layer to the antimicrobial foam article (52). In some examples, the adhesive layer may be the same as or similar to adhesive layer 30 as described above with reference to FIG. 3. With reference to FIG. 3, an adhesive layer may be optionally added to one or more of major surfaces 16, 18, or 24 of antimicrobial foam article 10. The adhesive layer may be applied by any technique known if the art including coating, lamination, immersion, or the like.

In some examples, the process of FIG. 6 optionally includes applying a film to the antimicrobial foam article (54). In some examples, the film may be the same as or similar to film 32 as described with reference to FIG. 3 above.

### Examples

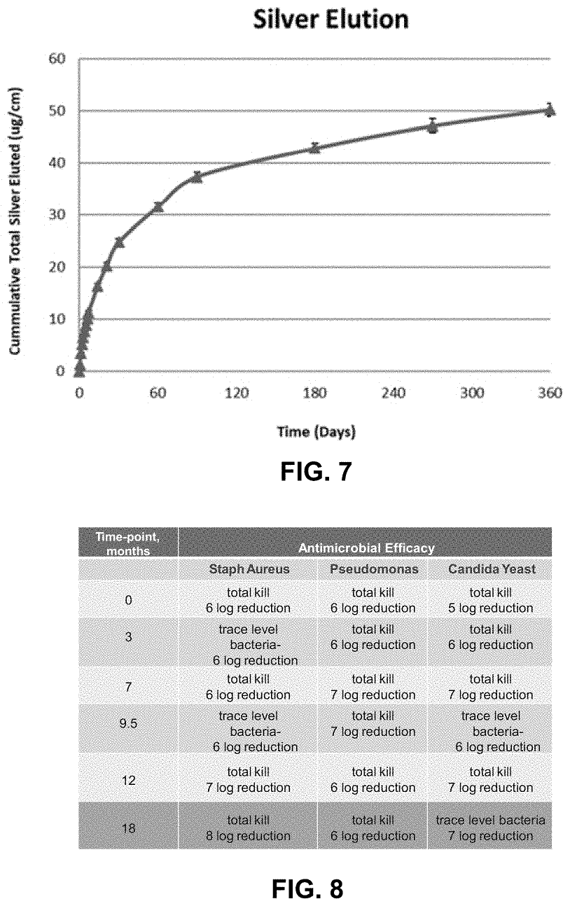

As noted above, in some examples, foam member 56 may exhibit a release profile that is suitable for a relatively long period of time. FIG. 7 is a chart showing the release profile of silver from an example antimicrobial formulation that may be used to form a foam member, such as foam member 56, in accordance with the present disclosure. The antimicrobial formulation used for the results of FIG. 7 included about 82 wt % polyurethane resin, about 7 wt % silver sulfadiazine, and about 11 wt % chlorhexidine acetate. As shown, active silver may be released from the article for at least 365 days (one year). Additionally, the cumulative total silver eluted may be greater than 40 μg/cm. As demonstrated in the chart, at least one antimicrobial agent encapsulated in a water permeable polymer may provide excellent antimicrobial efficacy because active antimicrobial agent may be eluted from the material over a long period of time, such as at least 5 days, or at least 10 days, or at least 50 days, or at least 100 days, or at least 150 days, or at least 200 days, or at least 250 days, or at least one year.

FIG. 8 is a chart illustrating the results of bacterial challenge testing for antimicrobial formulations that may be used to form an antimicrobial foam member, such as foam member 56, in accordance with the present disclosure. The antimicrobial formulation used to make antimicrobial foam articles in accordance with the present disclosure demonstrated antimicrobial efficacy over 18 months. Samples were aged in PBS at 37° C. and 50 rpm, with weekly change out of solution. Following aging, the samples were inoculated with microorganism. As demonstrated in the chart, at least one antimicrobial agent encapsulated in a water permeable polymer may provide excellent antimicrobial efficacy because active antimicrobial agent may be eluted from the material over a long period of time. Excellent antimicrobial efficacy may be measured by measuring the reduction of colonies of at least one microorganism.

In some examples, a sample may be inoculated with Staphylococcus aureus. After no aging, a total kill representing a 6 log reduction may be achieved. After three months of aging, only trace levels of bacteria may be present representing a 6 log reduction of colonies. After seven months of aging, a total kill representing a 6 log reduction of colonies may be achieved. After 9.5 months of aging, only trace levels of bacteria may be present representing a 6 log reduction of colonies. After 12 months of aging, a total kill representing a 7 log reduction of colonies may be achieved. After 18 months of aging, a total kill representing an 8 log reduction of colonies may be achieved. In some examples, a sample may be inoculated with Pseudomonas. After no aging, a total kill representing a 6 log reduction may be achieved. After three months of aging, a total kill representing a 6 log reduction may be achieved. After seven months of aging, a total kill representing a 7 log reduction of colonies may be achieved. After 9.5 months of aging, a total kill representing a 7 log reduction may be achieved. After 12 months of aging, a total kill representing a 6 log reduction of colonies may be achieved. After 18 months of aging, a total kill representing a 6 log reduction of colonies may be achieved. In some examples, a sample may be inoculated with Candida Yeast. After no aging, a total kill representing a 5 log reduction may be achieved. After three months of aging, a total kill representing a 6 log reduction may be achieved. After seven months of aging, a total kill representing a 7 log reduction of colonies may be achieved. After 9.5 months of aging, only trace levels of bacteria may be present representing a 6 log reduction of colonies. After 12 months of aging, a total kill representing a 7 log reduction of colonies may be achieved. After 18 months of aging, only trace levels of bacteria may be present representing a 7 log reduction of colonies.

Various embodiments of the invention have been described. These and other embodiments are within the scope of the following clauses and claims.

Clause 1. An antimicrobial article comprising a foam member including a polymer matrix and at least one antimicrobial agent encapsulated in a water permeable polymer, wherein the polymer matrix of the foam member defines a plurality of void volumes.

Clause 2. The antimicrobial article of clause 1, further comprising the antimicrobial agent within at least some of the void volumes of the polymer matrix.

Clause 3. The antimicrobial article of clause 1 or 2, wherein the at least one antimicrobial agent is partially encapsulated in the polymer matrix.

Clause 4. The antimicrobial article of any one of clauses 1-3, wherein the foam member includes at least about 10 weight percent of the at least one antimicrobial agent.

Clause 5. The antimicrobial article of any one of clauses 1-4, wherein the void fraction of the foam member is at least about 1 percent.

Clause 6. The antimicrobial article of clause 5, wherein the void fraction of the foam member is at least about 10 percent.

Clause 7. The antimicrobial article of any one of clauses 1-6, wherein the foam member an open-cell foam.

Clause 8. The antimicrobial article of any one of clauses 1-6, wherein the foam member is a closed-cell foam.

Clause 9. The antimicrobial article of any one of clauses 1-8, wherein the at least one antimicrobial agent is uniformly dispersed throughout the foam member.

Clause 10. The antimicrobial article of any one of clauses 1-9, wherein the at least one antimicrobial agent is uniformly dispersed throughout the foam member as particles and agglomerations of the antimicrobial agent having an average size of no greater than 50 microns.

Clause 11. The antimicrobial article of any one of clauses 1-10, wherein the article defines a contact surface configured to contact a target surface defined by a second article to provide an antimicrobial effect to the target surface.

Clause 12. The antimicrobial article of any one of clauses 1-11, further comprising an adhesive layer attached to the foam member.

Clause 13. The antimicrobial article of clause 12, wherein the adhesive layer extends beyond the edges of the foam member.

Clause 14. The antimicrobial article of any one of clauses 1-13, wherein the at least one water permeable polymer includes at least one of a polyurethane, thermoplastic polyurethan elastomer, polyester, polylactic acid, polyglycolic acid, polytetramethylene glycol, polyacrylamide, polyacrylic acid, polyacrylate, poly(2-hydoxy-ethyl methacrylate) polyethylene-imine, polysulfonate, or copolymers thereof.

Clause 15. The antimicrobial article of any one of clauses 1-14, wherein the at least one water permeable polymer has a weight average molecular weight of from about 70,000 to about 120,000 Daltons.

Clause 16. The antimicrobial article of any one of clauses 1-15, wherein the at least one antimicrobial agent comprises silver sulfadiazine, and wherein the foam member has a release profile such that silver sulfadiazine is released after 1 year.

Clause 17. The antimicrobial article of any one of clauses 1-16, wherein the at least one antimicrobial agent comprises chlorhexidine diacetate, and wherein the article has a release profile such that at least 10 μg/cm of chlorhexidine diacetate is released after 72 hours.

Clause 18. The antimicrobial article of any one of clauses 1-17, wherein the at least one antimicrobial agent comprises a silver-based salt and a polybiguanide salt.

Clause 19. The antimicrobial article of clause 18, wherein the silver-based salt is silver sulfadiazine and the polybiguanide salt is chlorhexidine diacetate.

Clause 20. The antimicrobial article of clause 19, wherein the article comprises from about 2 weight percent (wt. %) to about 10 wt. % of silver sulfadiazine, at least 9 wt. % of chlorhexidine diacetate and about 70 wt. % to about 90 wt. % of the at least one water permeable polymer.

Clause 21. The antimicrobial article of any one of clauses 1-20, wherein the at least one antimicrobial agent comprises silver sulfadiazine, and wherein the article continues to elute silver after 50 days.

Clause 22. The antimicrobial article of any one of clauses 1-21, wherein the at least one antimicrobial agent comprises silver sulfadiazine, and wherein the article continues to elute silver after 100 days.

Clause 23. A method of manufacturing an antimicrobial article, the method comprising forming a foam member from a polymer matrix and at least one antimicrobial agent encapsulated in a water permeable polymer matrix, wherein the polymer matrix of the foam member defines a plurality of void volumes.

Clause 24. The method of clause 23, further comprising encapsulating the at least one antimicrobial agent in the water permeable polymer which forms the polymer matrix.

Clause 25. The method of clause 24, wherein encapsulating the at least one antimicrobial agent includes milling at least one water permeable polymer in a liquid medium with at least one antimicrobial agent which is insoluble in the liquid medium.

Clause 26. The method of any one of clauses 23-25, wherein forming a foam further comprises pulling a vacuum to create a plurality of void volumes.

Clause 27. The method of any one of clauses 23-26, wherein forming a foam includes dissolving the water permeable polymer in a solvent.

Clause 28. The method of clause 27, wherein the solvent is tetrahydrofuran (THF).

Clause 29. The method of clause 25, wherein the milling media includes zirconia.

Clause 30. The method of any one of clauses 23-29, wherein forming the foam does not include adding a foaming agent or blowing agent.

Clause 31. The method of any one of clauses 23-30, wherein the foam member comprises uniformly dispersed particles of the at least one antimicrobial agent, wherein the particles and any agglomerations of the at least one antimicrobial agent have an average size of no greater than 50 microns.

Clause 32. The method of any one of clauses 23-31, wherein the at least one antimicrobial agent is partially encapsulated in the polymer matrix.

Clause 33. The method of any one of clauses 23-32, wherein the foam member includes at least about 10 weight percent of the at least one antimicrobial agent.

Clause 34. The method of any one of clauses 23-33, wherein the void fraction of the foam member is at least about 1 percent.

Clause 35. The method of clause 34, wherein the void fraction of the foam member is at least about 10 percent.

Clause 36. The method of any one of clauses 23-35, wherein the foam member an open-cell foam.

Clause 37. The method of any one of clauses 23-36, wherein the foam member is a closed-cell foam.

Clause 38. The method of any one of clauses 23-37, wherein the at least one antimicrobial agent is uniformly dispersed throughout the foam member.

Clause 39. The method of any one of clauses 23-38, wherein the antimicrobial agent is within at least some of the void volumes of the polymer matrix.

Clause 40. The method of any one of clauses 23-39, wherein the article defines a contact surface configured to contact a target surface defined by a second article to provide an antimicrobial effect to the target surface.

Clause 41. The method of any one of clauses 23-40, wherein the method further comprises attaching an adhesive layer attached to the foam member.

Clause 42. The antimicrobial article of clause 41, wherein the adhesive layer extends beyond the edges of the foam member.

Clause 43. The method of any one of clauses 23-42, wherein the at least one water permeable polymer includes at least one of a polyurethane, thermoplastic polyurethan elastomer, polyester, polylactic acid, polyglycolic acid, polytetramethylene glycol, polyacrylamide, polyacrylic acid, polyacrylate, poly(2-hydoxy-ethyl methacrylate) polyethylene-imine, polysulfonate, or copolymers thereof.

Clause 44. The method of any one of clauses 23-43, wherein the at least one water permeable polymer has a weight average molecular weight of from about 70,000 to about 120,000 Daltons.

Clause 45. The method of any one of clauses 23-44, wherein the at least one antimicrobial agent comprises silver sulfadiazine, and wherein the foam member has a release profile such that silver sulfadiazine is released after 1 year.

Clause 46. The method of any one of clauses 23-45, wherein the at least one antimicrobial agent comprises chlorhexidine diacetate, and wherein the article has a release profile such that at least 10 μg/cm of chlorhexidine diacetate is released after 72 hours.

Clause 47. The method of any one of clauses 23-46, wherein the at least one antimicrobial agent comprises a silver-based salt and a polybiguanide salt.

Clause 48. The method of clause 47, wherein the silver-based salt is silver sulfadiazine and the polybiguanide salt is chlorhexidine diacetate.

Clause 49. The method of clause 48, wherein the article comprises from about 2 weight percent (wt. %) to about 10 wt. % of silver sulfadiazine, at least 9 wt. % of chlorhexidine diacetate and about 70 wt. % to about 90 wt. % of the at least one water permeable polymer.

Clause 50. The method of any one of clauses 23-49, wherein the at least one antimicrobial agent comprises silver sulfadiazine, and wherein the article continues to elute silver after 50 days.

Clause 51. The method of any one of clauses 23-50, wherein the at least one antimicrobial agent comprises silver sulfadiazine, and wherein the article continues to elute silver after 100 days.

Clause 52. A method of using a wound dressing, the method comprising applying a wound dressing to a wound site, wherein the wound dressing comprises an antimicrobial article according to any one of clauses 1-22.

Clause 53. The method of clause 52, wherein applying the wound dressing includes packing a wound with the wound dressing.

Clause 54. The method of clauses 52 or 53, wherein applying the wound dressing includes fixing the wound dressing to the wound site with an adhesive.

Clause 55. The method of any one of clauses 52-54, comprising the further step of leaving the wound dressing in place without disturbance for at least 7 days.

1. An antimicrobial article comprising a foam member including a polymer matrix and at least one antimicrobial agent encapsulated in a water permeable polymer, wherein the polymer matrix of the foam member defines a plurality of void volumes.

2. The antimicrobial article of claim 1, further comprising the antimicrobial agent within at least some of the void volumes of the polymer matrix.

3. The antimicrobial article of claim 1, wherein the at least one antimicrobial agent is partially encapsulated in the polymer matrix.

4. The antimicrobial article of claim 1, wherein the foam member includes at least about 10 weight percent of the at least one antimicrobial agent.

5. The antimicrobial article of claim 1, wherein the void fraction of the foam member is at least about 1 percent.

6. The antimicrobial article of claim 5, wherein the void fraction of the foam member is at least about 10 percent.

7. The antimicrobial article of claim 1, wherein the foam member an open-cell foam.

8. The antimicrobial article of claim 1, wherein the foam member is a closed-cell foam.

9. The antimicrobial article of claim 1, wherein the at least one antimicrobial agent is uniformly dispersed throughout the foam member.

10. The antimicrobial article of claim 8, wherein the at least one antimicrobial agent is uniformly dispersed throughout the foam member as particles and agglomerations of the antimicrobial agent having an average size of no greater than 50 microns.

11. The antimicrobial article of claim 1, wherein the article defines a contact surface configured to contact a target surface defined by a second article to provide an antimicrobial effect to the target surface.

12. The antimicrobial article of claim 1, further comprising an adhesive layer attached to the foam member.

13. The antimicrobial article of claim 12, wherein the adhesive layer extends beyond edges of the foam member.

14. The antimicrobial article of claim 1, wherein the at least one antimicrobial agent comprises silver sulfadiazine, and wherein the foam member has a release profile such that silver sulfadiazine is released after 1 year.

15. The antimicrobial article of claim 1, wherein the at least one antimicrobial agent comprises chlorhexidine diacetate, and wherein the article has a release profile such that at least 10 μg/cm of chlorhexidine diacetate is released after 72 hours.

16. The antimicrobial article of claim 1, wherein the at least one antimicrobial agent comprises a silver-based salt and a polybiguanide salt.

17. The antimicrobial article of claim 1, wherein the polymer matrix includes the same polymer as the water permeable polymer.

18. A method of manufacturing an antimicrobial article, the method comprising forming a foam member from a polymer matrix and at least one antimicrobial agent encapsulated in a water permeable polymer matrix, wherein the polymer matrix of the foam member defines a plurality of void volumes.

19. The method of claim 18, further comprising encapsulating the at least one antimicrobial agent in the water permeable polymer prior to forming the foam member.

20. The method of claim 19, wherein encapsulating the at least one antimicrobial agent includes milling at least one water permeable polymer in a liquid medium with at least one antimicrobial agent which is insoluble in the liquid medium.
